(19) United States
(12) Patent Application Publication
(10) Pub. No.: US 2025/0154207 A1
(43) Pub. Date: May 15, 2025

(54) PEPTIDES FOR DISSOLVING PROTEIN AGGREGATES AND METHODS THEREOF

(71) Applicant: REJUKON BIOPHARM INC., Shanghai (CN)

(72) Inventor: Chenchen WANG, Shanghai (CN)

(21) Appl. No.: 18/838,613

(22) PCT Filed: Feb. 17, 2023
(86) PCT No.: PCT/CN2023/076790

§ 371 Date: Aug. 15, 2024

(30) Foreign Application Priority Data
Feb. 17, 2022 (WO) PCT/CN2022/076580

(51) Int. Cl. C07K 14/245; C12N 15/86

(52) U.S. Cl. CPC C07K 14/245; C12N 15/86; C12N 2750/14143

(57) ABSTRACT

The present disclosure provides a method of generating a peptide capable of dissolving protein aggregates. Also provided is a method of treating a neurodegeneration disease using such peptide.

### FIELD OF THE INVENTION

The present invention generally relates to neurobiology and neurodegenerative disease. In particular, the present invention relates to methods for generating peptides capable of dissolving pathogenic protein aggregates and methods of using such peptides.

### BACKGROUND OF THE INVENTION

Protein aggregation is a common feature of many neurodegenerative diseases, such as amyotrophic lateral sclerosis (ALS), Alzheimer's disease, Parkinson's disease and Huntington's disease. The protein aggregates in neurons, such as FUS in ALS, amyloid-beta in Alzheimer's disease, alpha-synuclein in Parkinson's disease and huntingtin in Huntington's disease, appear to be toxic, causing injury or death to neurons. In general, the degree of aggregation is proportional to the severity of the neurodegenerative disease.

While the causes of protein aggregation in neurons are not completely understood, it appears that the aggregation-prone proteins are often supersaturated in the cells, i.e., the cellular concentration of the protein exceeds the thermodynamic solubility but remain in a metastable liquid-like state by buffering via heterotypic interactions. Disturbance of metastable form of supersaturation would cause the loss of protein solubility and lead to protein aggregation.

Several attempts have been made to develop therapeutics for neurodegenerative diseases by inhibiting protein aggregation or dissolving pathogenic protein aggregates. For example, compounds capable of inhibiting protein aggregation have been disclosed in U.S. Pat. Nos. 10,435,373, 9,738,635, 10,889,584, 9,284,309, 9,527,852, and 9,790,188. U.S. Pat. No. 9,845,327 discloses inhibiting protein aggregation by promoting lysosomal activation. However, because there is still no known way to reverse the progressive degeneration of neurons, neurodegenerative diseases are considered as incurable. Therefore, there are needs to develop new compositions and methods to inhibit protein aggregation or dissolve protein aggregates in neurons.

### BRIEF SUMMARY OF THE INVENTION

In one aspect, the present disclosure provides a method of generating a peptide capable of dissolving a protein aggregate. In certain embodiments, the method comprises: identifying a naturally occurred protein having a charged amphipathic (Champ) region of 40 to 100 amino acid residues, wherein (a) at least 30 percent of the amino acid residues in the Champ region are charged amino acid residues selected from Asp, Glu, Arg and Lys, wherein at least 80 percent of the charged amino acid residues are in the N-terminus or C-terminus of the Champ region, (b) 14 percent to 26 percent of the amino acid residues in the Champ region are hydrophobic amino acid residues selected from Phe, Cys, Leu, Val and Ile, and (c) at least 10 percent of the amino acid residues in the Champ region are polar amino acid residues selected from Ser, Asn, Thr, Gln, Tyr, Cys and His, and generating a peptide consisting of the Champ region. In certain embodiments, the method further comprises: contacting the peptide with a protein aggregate; and determining that the peptide dissolves the protein aggregate. In certain embodiments, the method further comprises mutating any one of the charged amino acid residues in the peptide to a different a charged amino acid residue.

In another aspect, the present disclosure provides a peptide generated according to the method disclosed herein.

In another aspect, the present disclosure provides a polynucleotide encoding the peptide disclosed herein. In certain embodiments, the polynucleotide is a DNA or an RNA.

In yet another aspect, the present disclosure provides a vector comprising the polynucleotide disclosed herein. In certain embodiments, the vector is a virus vector, e.g., an AAV vector.

In another aspect, the present disclosure provides a recombinant virus comprising the polynucleotide disclosed herein. In certain embodiments, the recombinant virus is an AAV.

In another aspect, the present disclosure provides a host cell comprising the polynucleotide disclosed herein.

In another aspect, the present disclosure provides a pharmaceutical composition comprising (1) the peptide disclosed herein, the polynucleotide disclosed herein, the vector disclosed herein, the recombinant virus disclosed herein, or the host cell disclosed herein, and (2) a pharmaceutically acceptable carrier.

In yet another aspect, the present disclosure provides a method for dissolving a protein aggregate in a cell. In certain embodiments, the method comprises introducing into the cell the peptide disclosed herein. In certain embodiments, the cell is a neuronal cell. In certain embodiments, the cell is in vitro or in vivo.

In another aspect, the present disclosure provides a method for treating a neurodegeneration disease in a subject in need thereof. In certain embodiments, the method comprises administering to the subject a therapeutic effective amount of the pharmaceutical composition disclosed herein. In certain embodiments, the neurodegeneration disease is selected from frontotemporal dementia, amyotrophic lateral sclerosis, cortical basal ganglia degeneration, Lewy body dementia, Huntington's disease, Lewy body disease, motor neuron disease, frontotemporal degeneration, hippocampal sclerosis, inclusion body myopathy, inclusion body Myositis, Parkinson's disease, argyrophilic granular disease, Alzheimer's disease, Parsons/dementia complex in the Kii Peninsula, progressive supranuclear palsy, and Pick's disease. In certain embodiments, the pharmaceutical composition is administered to the central nervous system. In certain embodiments, the pharmaceutical composition is administered via spinal cord injection, intrathecal injection, intracerebroventricular injection, intracerebral injection, or intra-hippocampal injection.

### BRIEF DESCRIPTION OF THE FIGURES

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the distribution of different type of amino acid residues in a peptide Champ-E that has depolymerization effect.

FIG. 2 shows the sequences of series of variant peptides derived from Champ-E.

FIG. 3 shows the Coomassie blue staining of Champ-E variants purified from recombinant E coli.

FIG. 4 shows the effect of Champ-E variants in dissolving G3BP1 aggregates.

FIG. 5 shows the effect of Champ-E variants in dissolving FDA.

FIG. 6 shows the experimental design of identifying peptides having depolymerization effect from eukaryotic cells.

FIG. 7 shows the depolymerization effect of PLPR3 peptide in eukaryotic cells.

FIG. 8 shows the depolymerization effect of ATXN80S peptide in eukaryotic cells.

FIG. 9 shows the depolymerization effect of GRWD1 peptide in eukaryotic cells.

FIG. 10 shows the depolymerization effect of TSYL2 peptide in eukaryotic cells.

FIG. 11 shows the depolymerization effect of C19L2 peptide in eukaryotic cells.

FIG. 12 shows the depolymerization effect of ZNF326 peptide in eukaryotic cells.

FIG. 13 shows the depolymerization effect of CACNA1F peptide in eukaryotic cells.

FIG. 14 shows the depolymerization effect of variant Champ6EK peptide in eukaryotic cells.

FIG. 15 shows the depolymerization effect of CHD3 peptide in eukaryotic cells.

FIG. 16 shows the depolymerization effect of variant CHD3(E2K) peptide in eukaryotic cells.

FIG. 17 shows the depolymerization effect of PELP1 peptide in vivo.

FIG. 18 shows the amino acid codes.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definition

It is to be understood that both the foregoing general description and the following detailed description are exemplary and explanatory only and are not restrictive of the invention as claimed. In this application, the use of the singular includes the plural unless specifically stated otherwise. In this disclosure, the term “or” is used to mean “and/or” unless explicitly indicated to refer to alternatives only or the alternatives are mutually exclusive. As used herein “another” may mean at least a second or more. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

As used herein, the singular forms “a”, “an” and “the” include plural references unless the context clearly dictates otherwise.

As used herein, the term “administering” means providing a pharmaceutical agent or composition to a subject, and includes, but is not limited to, administering by a medical professional and self-administering.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure.

A “cell”, as used herein, can be prokaryotic or eukaryotic. A prokaryotic cell includes, for example, bacteria. A eukaryotic cell includes, for example, a fungus, a plant cell, and an animal cell. Preferably, the cell described herein is an animal cell. The types of an animal cell (e.g., a mammalian cell or a human cell) includes, for example, a cell from a nervous system or organ, e.g., a neuron, a glioblast (e.g., astrocyte and oligodendrocyte), a microglia, a magnocellular neurosecretory cell, a stellate cell, a boettcher cell, and a pituitary cell (e.g., gonadotrope, corticotrope, thyrotrope, somatotrope, and lactotroph); a cell from circulatory/immune system or organ, e.g., a B cell, a T cell (cytotoxic T cell, natural killer T cell, regulatory T cell, T helper cell), a natural killer cell, a granulocyte (e.g., basophil granulocyte, an eosinophil granulocyte, a neutrophil granulocyte and a hypersegmented neutrophil), a monocyte or macrophage, a red blood cell (e.g., reticulocyte), a mast cell, a thrombocyte or megakaryocyte, and a dendritic cell; a cell from an endocrine system or organ, e.g., a thyroid cell (e.g., thyroid epithelial cell, parafollicular cell), a parathyroid cell (e.g., parathyroid chief cell, oxyphil cell), an adrenal cell (e.g., chromaffin cell), and a pineal cell (e.g., pinealocyte); a cell from a respiratory system or organ, e.g., a pneumocyte (a type I pneumocyte and a type II pneumocyte), a clara cell, a goblet cell, an alveolar macrophage; a cell from circular system or organ, e.g., myocardiocyte and pericyte; a cell from digestive system or organ, e.g., a gastric chief cell, a parietal cell, a goblet cell, a paneth cell, a G cell, a D cell, an ECL cell, an I cell, a K cell, an S cell, an enteroendocrine cell, an enterochromaffin cell, an APUD cell, a liver cell (e.g., a hepatocyte and Kupffer cell); a cell from integumentary system or organ, e.g., a bone cell (e.g., an osteoblast, an osteocyte, and an osteoclast), a teeth cell (e.g., a cementoblast, and an ameloblast), a cartilage cell (e.g., a chondroblast and a chondrocyte), a skin/hair cell (e.g., a trichocyte, a keratinocyte, and a melanocyte (Nevus cell), a muscle cell (e.g., myocyte), an adipocyte, a fibroblast, and a tendon cell), a cell from urinary system or organ (e.g., a podocyte, a juxtaglomerular cell, an intraglomerular mesangial cell, an extraglomerular mesangial cell, a kidney proximal tubule brush border cell, and a macula densa cell), and a cell from reproductive system or organ (e.g., a spermatozoon, a Sertoli cell, a leydig cell, an ovum, an oocyte). A cell can be normal, healthy cell; or a diseased or unhealthy cell (e.g., a cancer cell). A mammalian cell can be a rodent cell, e.g., a mouse, rat, hamster cell. A mammalian cell can be a lagomorpha cell, e.g., a rabbit cell. A mammalian cell can also be a primate cell, e.g., a human cell

As used herein, the term “effective amount” or “therapeutically effective amount” means the amount of agent that is sufficient to prevent, treat, reduce and/or ameliorate the symptoms and/or underlying causes of any disorder or disease, or the amount of an agent sufficient to produce a desired effect on a cell. In one embodiment, a “therapeutically effective amount” is an amount sufficient to reduce or eliminate a symptom of a disease. In another embodiment, a therapeutically effective amount is an amount sufficient to overcome the disease itself.

The term “host cell” means a cell that has been transformed, or is capable of being transformed, with a nucleic acid sequence and thereby expresses a protein of interest. The term includes the progeny of the parent cell, whether or not the progeny is identical in morphology or in genetic make-up to the original parent cell, so long as the gene of interest is present.

The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23(21): 2947-8 (2007)), and ALIGN or Megalign (DNASTAR) software. A person skilled in the art may use the default parameters provided by the tool or may customize the parameters as appropriate for the alignment, such as for example, by selecting a suitable algorithm.

The term “polypeptide” or “protein” means a string of at least two amino acids linked to one another by peptide bonds. Polypeptides and proteins may include moieties in addition to amino acids (e.g., may be glycosylated) and/or may be otherwise processed or modified. Those of ordinary skill in the art will appreciate that a “polypeptide” or “protein” can be a complete polypeptide chain as produced by a cell (with or without a signal sequence) or can be a functional portion thereof. Those of ordinary skill will further appreciate that a polypeptide or protein can sometimes include more than one polypeptide chain, for example linked by one or more disulfide bonds or associated by other means. The term also includes amino acid polymers in which one or more amino acids are chemical analogs of a corresponding naturally occurring amino acid and polymers.

The phrase “pharmaceutically acceptable carrier” as used herein means a pharmaceutically acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “protein aggregate” used herein refers the aggregation of a protein which appears either intra or extracellularly. In some embodiments, the protein is an intrinsically disordered protein or a mis-folded protein. In some embodiments, the aggregation occurs when the concentration of the protein exceeds the solubility of the protein. In some embodiments, the concentration of the protein exceeds the thermodynamic solubility, but the protein remains in a metastable liquid-like state by buffering via heterotypic interactions. Disturbance of metastable form of the protein would cause the loss of protein solubility and lead to protein aggregation.

The term “recombinant” when used with reference to a polypeptide (e.g., antibody, antigen) or a polynucleotide, refers to a polypeptide or polynucleotide that is produced by a recombinant method. A “recombinant polypeptide” includes any polypeptide expressed from a recombinant polynucleotide. A “recombinant polynucleotide” includes any polynucleotide which has been modified by the introduction of at least one exogenous (i.e., foreign, and typically heterologous) nucleotide or the alteration of at least one native nucleotide component of the polynucleotide and need not include all of the coding sequence or the regulatory elements naturally associated with the coding sequence. A “recombinant vector” refers to a non-naturally occurring vector, including, e.g., a vector comprising a recombinant polynucleotide sequence.

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

As used herein, a “vector” refers to a nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

### Methods of Generating Aggregate-Dissolving Peptide

Protein aggregation is a biological phenomenon in which intrinsically disordered proteins or mis-folded proteins aggregate (i.e., accumulate and clump together) either intra- or extracellularly. Protein structures are stabilized by non-covalent interactions and disulfide bonds between two cysteine residues. The non-covalent interactions include ionic interactions and weak van der Waals interactions. Ionic interactions form between an anion and a cation and form salt bridges that help stabilize the protein. Van der Waals interactions include nonpolar interactions and polar interactions (i.e., hydrogen bonds, dipole-dipole bond). These play an important role in a protein's secondary structure, such as forming an alpha helix or a beta sheet, and tertiary structure. Interactions between amino acid residues in a specific protein are very important in that protein's final structure.

When there are changes in the non-covalent interactions, as may happen with a change in the amino acid sequence, the protein is susceptible to misfolding or unfolding. In these cases, if the cell does not assist the protein in re-folding, or degrade the unfolded protein, the unfolded/misfolded protein may aggregate, in which the exposed hydrophobic portions of the protein may interact with the exposed hydrophobic patches of other proteins. There are three main types of protein aggregates that may form: amorphous aggregates, oligomers, and amyloid fibrils. Mis-folded protein aggregates are often correlated with diseases. For example, protein aggregates have been implicated in a wide variety of neurodegenerative diseases, including ALS, Alzheimer's disease, Parkinson's disease, Huntington's disease and prion disease.

The present disclosure in one aspect provides a method for identifying a polypeptide capable of dissolving a protein aggregate. In certain embodiments, the method comprises: identifying a naturally occurred protein having a charged amphipathic (Champ) region of 40 to 100 amino acid residues, wherein (a) at least 30 percent of the amino acid residues in the Champ region are charged amino acid residues selected from Asp, Glu, Arg and Lys, wherein at least 80 percent of the charged amino acid residues are in the N-terminus or C-terminus of the Champ region, (b) 14 percent to 26 percent of the amino acid residues in the Champ region are hydrophobic amino acid residues selected from Phe, Cys, Leu, Val and Ile, and (c) at least 10 percent of the amino acid residues in the Champ region are polar amino acid residues selected from Ser, Asn, Thr, Gln, Tyr, Cys and His, and generating a peptide consisting of the Champ region.

In certain embodiments, the method further comprises: contacting the peptide with a protein aggregate; and determining that the peptide dissolves the protein aggregate. In certain embodiments, the protein aggregate is FUS aggregate, TDP43 aggregate, TIA1 aggregate, and C9orf72 aggregate. In some embodiment, the peptide is capable of dissolving at least 10%, 20%, 30%, 40%, 50%, 60%, 70%, 80%, 90% of the protein aggregate.

Variants of the polypeptide can be generated by replacing amino acid residues in the polypeptide such that the variant has a sequence of at least 90% identity to the polypeptide, or a sequence having 1, 2, 3, 4, or 5 amino acid residue difference from the polypeptide. The variants generated can then be tested for its ability to dissolve protein aggregates. In some embodiment, the variant has an improved capability of dissolving the protein aggregate compared to the polypeptide from which the variant is derived. In certain embodiments, the method further comprises mutating any one of the charged amino acid residues in the peptide to a different a charged amino acid residue.

### Compositions for Dissolving Protein Aggregates

The present disclosure in another aspect provides a peptide capable of dissolving protein aggregates. In some embodiments, the peptide is generated according to the method disclosed herein.

In another aspect, the present disclosure provides a polynucleotide encoding the peptide described herein. In some embodiments, the polynucleotide is a DNA or an RNA. In some embodiments, the polynucleotide is single strand DNA or double strand DNA.

In another aspect, the present disclosure provides a vector comprising the polynucleotide disclosed herein. Typically, the vector further comprises additional elements that facilitate the expression of the polypeptide, such as promoter, enhancer, polyA region, etc. In some embodiments, the vector is a virus vector. In some embodiments, the vector is an adeno-associated virus (AAV) vector. In some embodiments, the AAV vector further comprises an ITR sequence.

In another aspect, the present disclosure provides a recombinant virus comprising the polynucleotide disclosed herein. In some embodiments, the recombinant virus is an AAV. In some embodiments, the AAV has a serotype selected from AAV1, AAV2, AAV3, AAV5, AAV6, AAV7, AAV8, AAVrh8, AAV9, AAV10, AAVrh10, AAV11 and AAV12.

In another aspect, the present disclosure provides a host cell comprising the polynucleotide disclosed herein. The host cell can be used to express the peptide disclosed herein or to generate the virus disclosed herein.

In another aspect, the present disclosure provides pharmaceutical compositions comprising the peptide, the polynucleotide, the vector, the recombinant virus, or the host cell disclosed herein. Such compositions comprise a prophylactically or therapeutically effective amount of the peptide, the polynucleotide, the vector, the recombinant virus, or the host cell, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical agents are described in “Remington's Pharmaceutical Sciences.” Such compositions will contain a prophylactically or therapeutically effective amount of the polypeptide, the polynucleotide, the vector, the recombinant virus, or the host cell, together with a suitable amount of carrier so as to provide the form for proper administration to the patient. The formulation should suit the mode of administration, which can be intrathecal, intravenous, intraarterial, intrabuccal, intranasal, nebulized, bronchial inhalation, or delivered by mechanical ventilation. Where the composition is administered by injection, an ampoule of sterile water for injection or saline can be provided so that the ingredients may be mixed prior to administration.

The compositions of the present disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### METHODS OF USE

In another aspect, the present disclosure provides a method of dissolving protein aggregates in a cell. In some embodiments, the method comprises introducing into the cell the peptide disclosed herein. In some embodiments, the cell is a neuron. In some embodiments, the protein aggregate is a FUS protein aggregate, a TDP-43 protein aggregate, a TIA1 protein aggregate, a beta-amyloid protein aggregate, an alpha-synuclein protein aggregate, or a huntingtin protein aggregate. In some embodiments, the polypeptide is introduced into the cell by contacting the cell with the peptide. In some embodiments, the peptide is introduced into the cell by introducing a polynucleotide encoding the peptide disclosed herein into the cell, thus allowing the cell to express the peptide. In some embodiments, the polynucleotide is introduced into the cell by transforming or transfecting the cell with a vector or virus comprising the polynucleotide.

In yet another aspect, the present disclosure provides a method of treating a disease or condition related to protein aggregation. In some embodiments, the disease or condition related to protein aggregation is a neurodegenerative disease. Neurodegenerative diseases result in the progressive loss of structure or function, and ultimately the cell death of neurons. Examples of neurodegenerative diseases include, without limitation, frontotemporal dementia, amyotrophic lateral sclerosis (ALS), cortical basal ganglia degeneration, Lewy body dementia, Huntington's disease, Lewy body disease, motor neuron disease, frontotemporal degeneration, hippocampal sclerosis, inclusion body myopathy, inclusion body Myositis, Parkinson's disease, argyrophilic granular disease, Alzheimer's disease, Parsons/dementia complex in the Kii Peninsula, progressive supranuclear palsy, and Pick's disease.

Amyotrophic lateral sclerosis (ALS) involves the degeneration of the upper motor neurons (UMNs) and lower motor neurons (LMNs) with the symptom of gradual progress of skeletal muscle weakness. Missense mutations in the gene encoding the antioxidant enzyme Cu/Zn superoxide dismutase 1 (SOD1) were discovered in a subset of patients with familial ALS. However, the pathogenic mechanism underlying SOD1 mutant toxicity has yet to be resolved. TDP-43 and FUS protein aggregates have also been implicated in some cases of the disease, and a mutation in chromosome 9 (C9orf72) is thought to be the most commonly known cause of sporadic ALS.

Alzheimer's disease (AD) is a chronic neurodegenerative disease that results in the loss of neurons and synapses in the cerebral cortex and certain subcortical structures, resulting in gross atrophy of the temporal lobe, parietal lobe, and parts of the frontal cortex and cingulate gyrus. AD pathology is primarily characterized by the presence of senile plaques and neurofibrillary tangles. Plaques are made up of small peptides, typically 39-43 amino acids in length, called beta-amyloid (also written as A-beta or AR). Beta-amyloid is a fragment from a larger protein called amyloid precursor protein (APP), a transmembrane protein that penetrates through the neuron's membrane. APP appears to play roles in normal neuron growth, survival and post-injury repair. APP is cleaved into smaller fragments by enzymes such as gamma secretase and beta secretase. One of these fragments gives rise to fibrils of beta-amyloid which can self-assemble into the dense extracellular deposits known as senile plaques or amyloid plaques.

Parkinson's disease (PD) is the second most common neurodegenerative disorder. It typically manifests as bradykinesia, rigidity, resting tremor and posture instability. PD is primarily characterized by death of dopaminergic neurons in the substantia nigra, a region of the midbrain. The cause of this selective cell death is unknown. Notably, alpha-synuclein-ubiquitin complexes and aggregates are observed to accumulate in Lewy bodies within affected neurons. It is thought that defects in protein transport machinery and regulation, such as RAB1, may play a role in this disease mechanism. Impaired axonal transport of alpha-synuclein may also lead to its accumulation in Lewy bodies. Experiments have revealed reduced transport rates of both wild-type and two familial Parkinson's disease-associated mutant alpha-synucleins through axons of cultured neurons.

Huntington's disease (HD) is a rare autosomal dominant neurodegenerative disorder caused by mutations in the huntingtin gene (HTT). HD is characterized by loss of medium spiny neurons and astrogliosis. The first brain region to be substantially affected is the striatum, followed by degeneration of the frontal and temporal cortices. The striatum's subthalamic nuclei send control signals to the globus pallidus, which initiates and modulates motion. The weaker signals from subthalamic nuclei thus cause reduced initiation and modulation of movement, resulting in the characteristic movements of the disorder, notably chorea. HD is caused by polyglutamine tract expansion in the huntingtin gene, resulting in the mutant huntingtin. Aggregates of mutant huntingtin form as inclusion bodies in neurons and may be directly toxic. Additionally, they may damage molecular motors and microtubules to interfere with normal axonal transport, leading to impaired transport of important cargoes such as BDNF.

In some embodiments, the method of treating a disease or condition related to protein aggregation comprises administering to a subject in need thereof a therapeutic effective amount of the pharmaceutical composition disclosed herein.

The therapeutically effective amount of the pharmaceutical composition provided herein will depend on various factors known in the art, such as for example type of disease to be treated, body weight, age, past medical history, present medications, state of health of the subject, immune condition and potential for cross-reaction, allergies, sensitivities and adverse side-effects, as well as the administration route and the type, the severity and development of the disease and the discretion of the attending physician or veterinarian. In certain embodiments, the pharmaceutical composition provided herein may be administered at a therapeutically effective dosage of about 0.001 mg/kg to about 100 mg/kg one or more times per day (e.g., about 0.001 mg/kg, about 0.3 mg/kg, about 0.5 mg/kg, about 1 mg/kg, about 3 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg one or more times per day). In certain embodiments, the pharmaceutical composition is administered at a dosage of about 50 mg/kg or less, and in certain embodiments the dosage is 20 mg/kg or less, 10 mg/kg or less, 3 mg/kg or less, 1 mg/kg or less, 0.3 mg/kg or less, 0.1 mg/kg or less, or 0.01 mg/kg or less, or 0.001 mg/kg or less. In certain embodiments, the administration dosage may change over the course of treatment. For example, in certain embodiments the initial administration dosage may be higher than the subsequent administration dosages. In certain embodiments, the administration dosage may vary over the course of treatment depending on the reaction of the subject.

Dosage regimens may be adjusted to provide the optimum desired response (e.g., a therapeutic response). In certain embodiments, the pharmaceutical composition provided herein is administered to the subject at one time or over a series of treatments. In certain embodiments, the pharmaceutical composition provided herein is administered to the subject by one or more separate administrations, or by continuous infusion depending on the type and severity of the disease.

The pharmaceutical composition provided herein may be administered by any route known in the art, such as for example parenteral (e.g., subcutaneous, intraperitoneal, intravenous, including intravenous infusion, intramuscular, intradermal or intrathecal injection) or non-parenteral (e.g., oral, intranasal, intraocular, sublingual, rectal, or topical) routes.

In some embodiments, the pharmaceutical composition is administered to the central nervous system. In some embodiments, the pharmaceutical composition is administered via spinal cord injection, intrathecal injection, intracerebroventricular injection, intracerebral injection, or intra-hippocampal injection. In some embodiments, the pharmaceutical composition provided herein is administered by intrathecal routes. As used herein, the terms “intrathecal administration,” “intrathecal injection,” “intrathecal delivery,” or grammatic equivalents, refer to an injection into the spinal canal (intrathecal space surrounding the spinal cord). In some embodiments, “intrathecal administration” or “intrathecal delivery” according to the present disclosure refers to IT administration or delivery via the lumbar area or region (i.e., lumbar IT administration or delivery,) or cisterna magna delivery (i.e., injection via the space around and below the cerebellum via the opening between the skull and the top of the spine).

The following examples are provided to better illustrate the claimed invention and are not to be interpreted in any way as limiting the scope of the invention. All specific compositions, materials, and methods described below, in whole or in part, fall within the scope of the invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the invention. It is the intention of the inventors that such variations are included within the scope of the invention.

TABLE 1
Sequences mentioned in the application

| SEQ | | |
|---|---|---|
| ID | | |
| NO: | Amino acid sequence | Name |
| 1 | MTEPQEESEEEVEEPEERQQT | Champ-E |
| | PEVVPDDSGTFYDQTVSNDLE | |
| 2 | TEPQEESEEEVEEPEERQQTP | Champ-E′ |
| | EVVPDDSGTFYDQTVSNDLE | |
| 3 | TDPQDDSDDDVDDPDDRQQTP | Champ-D |
| | DVVPDDSGTFYDQTVSNDLD | |
| 4 | TKPQKKSKKKVKKPKKRQQTP | Champ-K |
| | KVVPDDSGTFYDQTVSNDLK | |
| 5 | TQPQQQSQQQVQQPQQRQQTP | Champ-Q |
| | QVVPDDSGTFYDQTVSNDLQ | |
| 6 | PMAEEEEEEEDEEEEEEEEEE | PLPR3 |
| | EDEGPAPPSLYPTVQARPG | |
| 7 | PCPGAPCCSLVATGSRVPFSG | ATXN8OS |
| | LKEEEEEDGEDDEEEEEEG | |
| 8 | SNRLMMLRMHNLHGTKPPPSE | GRWD1 |
| | GSDEEEEEEDEEDEEERKP | |
| 9 | RSSRRRRRRRRRKQRKVKRES | TSYL2 |
| | RERNAERMESILQALEDIQ | |
| 10 | LPDVNERIEQFSQEHSVKKKK | C19L2 |
| | KKDKHSKKAKKEKKKKSKK | |
| 11 | DEPIEEEEDEDEEEEAEEVGE | ZNF326 |
| | VEEVEEVEEVREGGIEGEG | |
| 12 | DMEEEEEEEEEEEEEEEEEGA | CACNA1F |
| | GGVELLQEVVPKEKVVPIP | |
| 13 | REKKEKKTKRRKKGEGDGGQK | CHD3 |
| | QVEQKSSATLLLTWGLEDV | |
| 14 | RKKKKKKTKRRKKGKGDGGQK | CHD3(E2K) |
| | QVKQKSSATLLLTWGLKDV | |
| 15 | GGPPALEEDLTVININSSDEE | PELP1 |
| | EEEEEEEEEEEEEEEEEEE | |

### Example 1

This example illustrates the analysis of a charged amphipathic (Champ) peptide capable of dissolving protein aggregates.

The inventors have previously identified a Champ peptide (Champ-E) capable of dissolving protein aggregation, which is disclosed in PCT application PCT/CN2021/111683 and has the amino acid sequence of SEQ ID NO: 1.

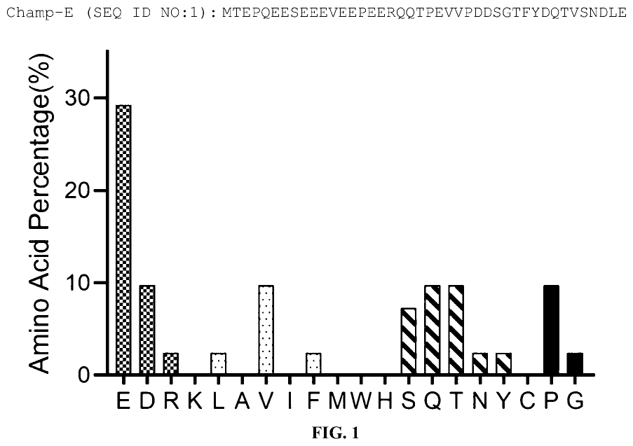

The inventors analyzed the percentage of different types of amino acid residues in Champ-E and the distribution of the amino acid residues. The result of the analysis is shown in FIG. 1. As shown in FIG. 1, 41.5% of the amino acid residues in Champ-E are charged amino acid residues (E/D/R/K), 17.1% of the amino acid residues in Champ-E are hydrophobic amino acid residues (L/A/V/I/F/M/W), 29.3% of the amino acid residues in Champ-E are polar amino acid residues (H/S/Q/T/N/Y/C), 12.2% of the amino acid residues in Champ-E are Proline or Glycine (P/G).

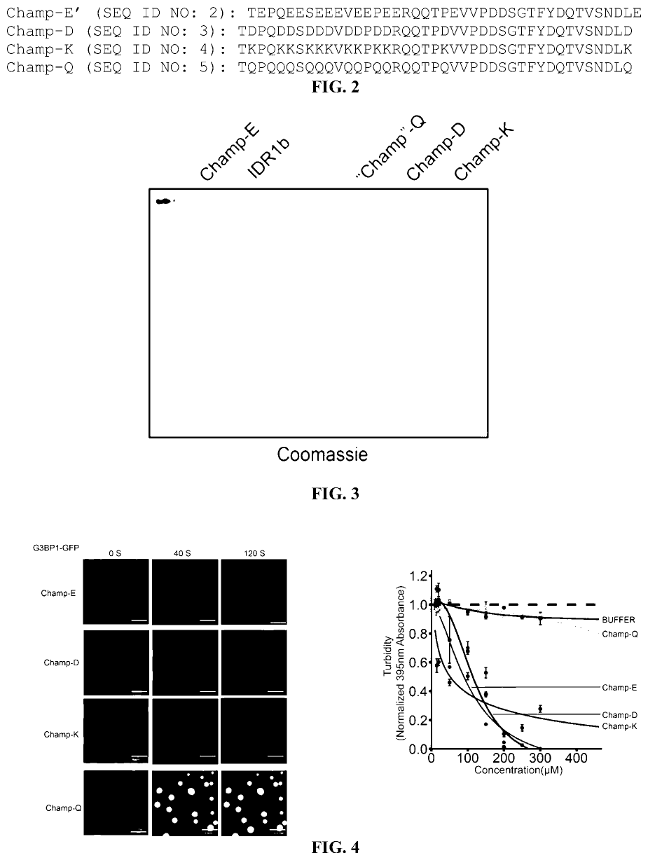

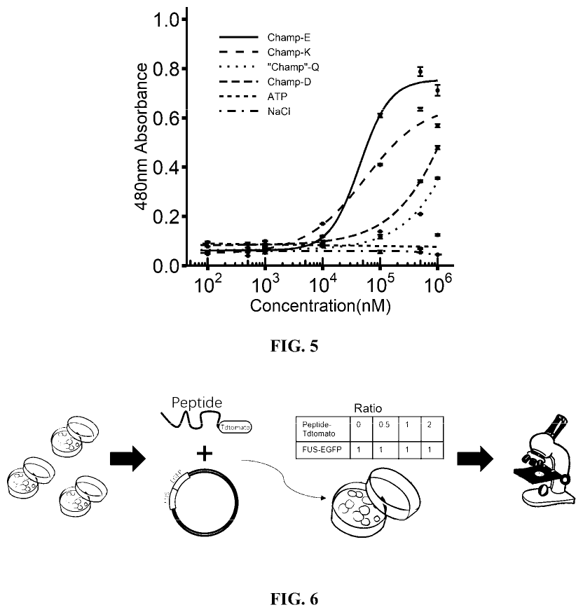

Given that the charged amino acid residues have the highest percentage in Champ-E, the inventors first tested the function of the charged amino acid residues in dissolving protein aggregates. The inventors generated a series variant of Champ-E with mutation of aspartic acid residues and tested their capability of dissolving protein aggregates in vitro and in cells. The sequences of the variant peptides are shown in FIG. 2. The variant peptides were expressed in E. coli and then purified (see FIG. 3). The purified variant peptides were then added to in vitro protein solution that is capable of forming protein aggregates. The inventors found that charged variant peptides, no matter positively charged or negatively charged, were able to dissolve protein aggregates (see FIG. 4). On the other hand, the “Champ”-Q variant peptide, which is not charged, could no longer dissolve protein aggregates.

As the charged amino acid residues in Champ-E are more concentrated at the N-terminus and the NMR data in the process of dissolving protein aggregates show that the charge change at the N-terminus is relatively higher (results not shown), the inventors speculated that amphipathic properties of the peptide may be important for dissolving protein aggregates. The inventors used in vitro FDA (Fluorescein diacetate, which has no absorption light in insoluble state, but has absorption light at 488 nm wavelength in soluble state) analysis to identify the amphiphilicity of different short peptides and the solubility of FDA dyes. As shown in FIG. 5, the results showed that the charged short peptides Champ-E/Champ-D/Champ-K all had strong solubility, which is 100 times higher than that of the positive control ATP, while “Champ”-Q, which was mutated to polar amino acids and has no obvious depolymerization effect in vitro, has the worst solubility. The results indicated that amphiphilicity is also necessary for the depolymerization function.

### Example 2

This example illustrates the generation of Champ peptides capable of dissolving protein aggregates.

Based on the known characteristics of Champ-E and short peptides that still have depolymerization effect after being mutated to different charges, as well as the influence of in vitro amphipathic characteristics on the depolymerization effect, the inventors screened the human proteomics to identify peptides of 40 amino acid residues (such peptide is a fragment of a naturally occurred human protein) based on the following standard: (a) at least 30% of the amino acid residues in the peptide are (i) charged amino acid residues selected from Asp and Glu, or (ii) charged amino acid residues selected from Arg and Lys, wherein at least 80 percent of the charged amino acid residues are in the N-terminus or C-terminus of the peptide, (b) 14%-26% of the amino acid residues in the peptide are hydrophobic amino acid residues selected from Phe, Cys, Leu, Val and Ile, and (c) at least 10% of the amino acid residues in the peptide are polar amino acid residues selected from Ser, Asn, Thr, Gln, Tyr, Cys and His. A total of 13,543 sequences of peptides were identified.

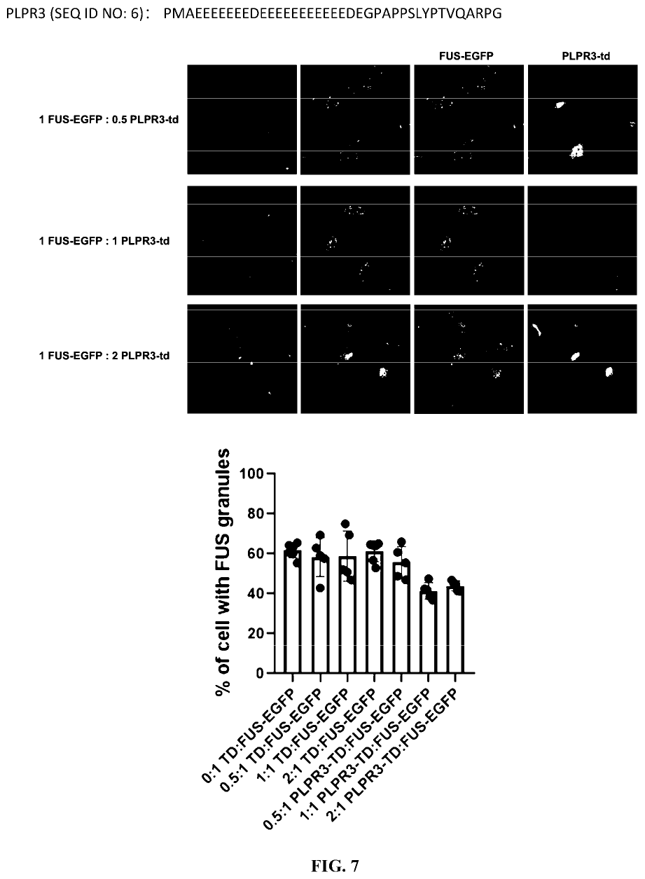

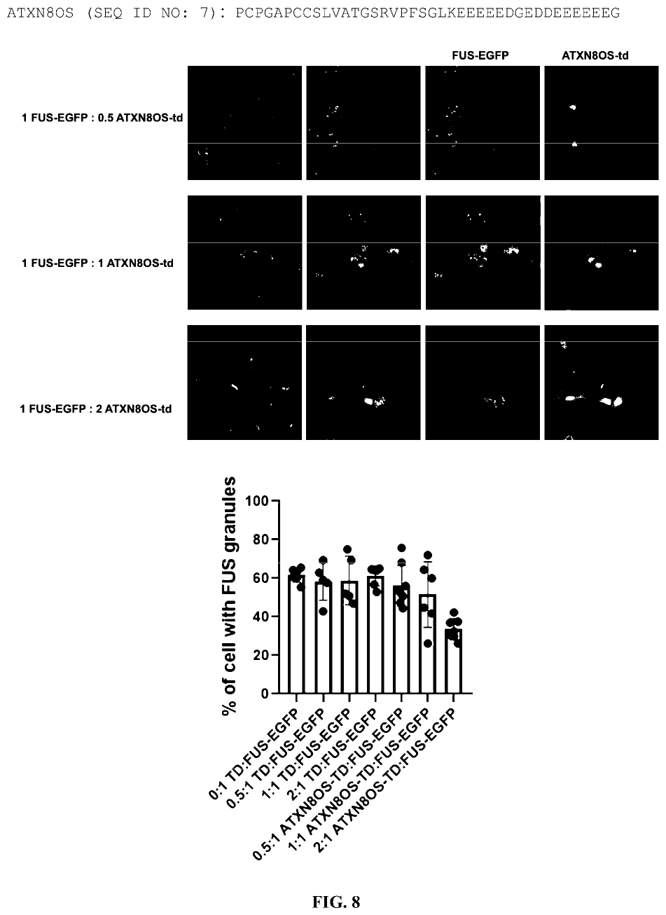

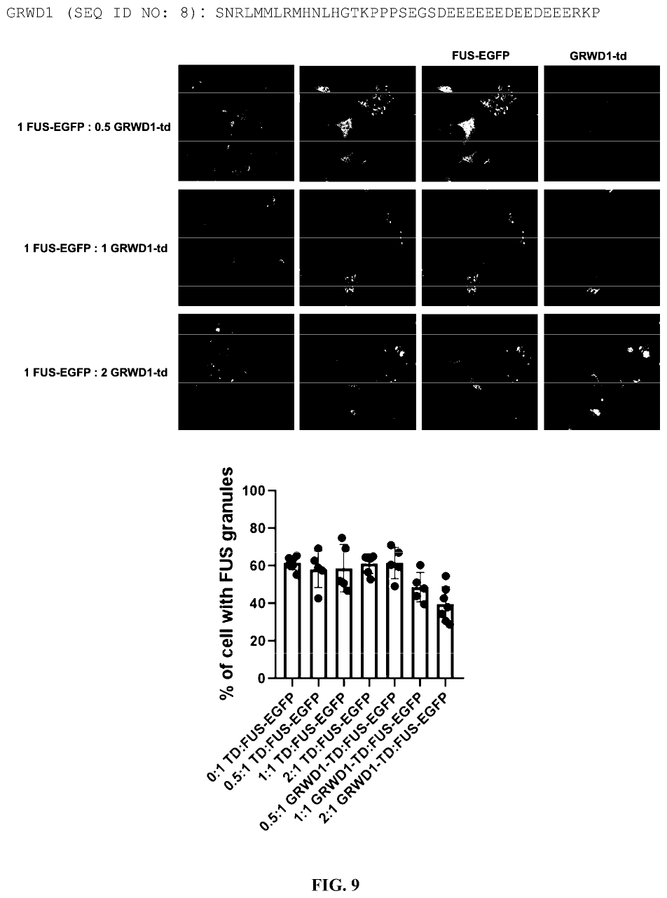

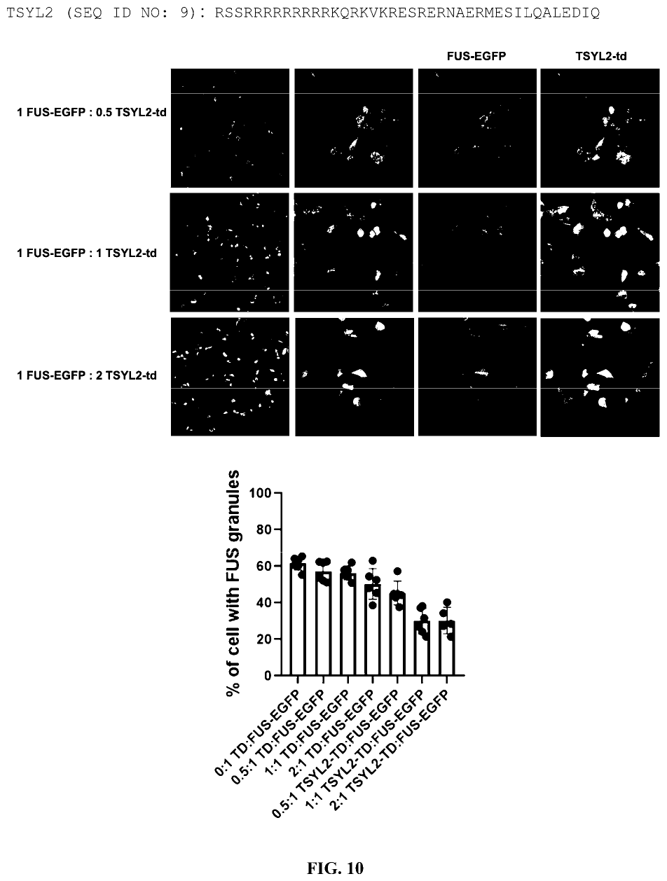

The inventors then selected three peptides PLPR3 (SEQ ID NO: 6), ATXN8OS (SEQ ID NO: 7), and GRWD1 (SEQ ID NO: 8) to verify the depolymerization function in eukaryotic cells with the experimental scheme shown in FIG. 6. As shown in FIGS. 7-9, all three selected peptides have depolymerization effect.

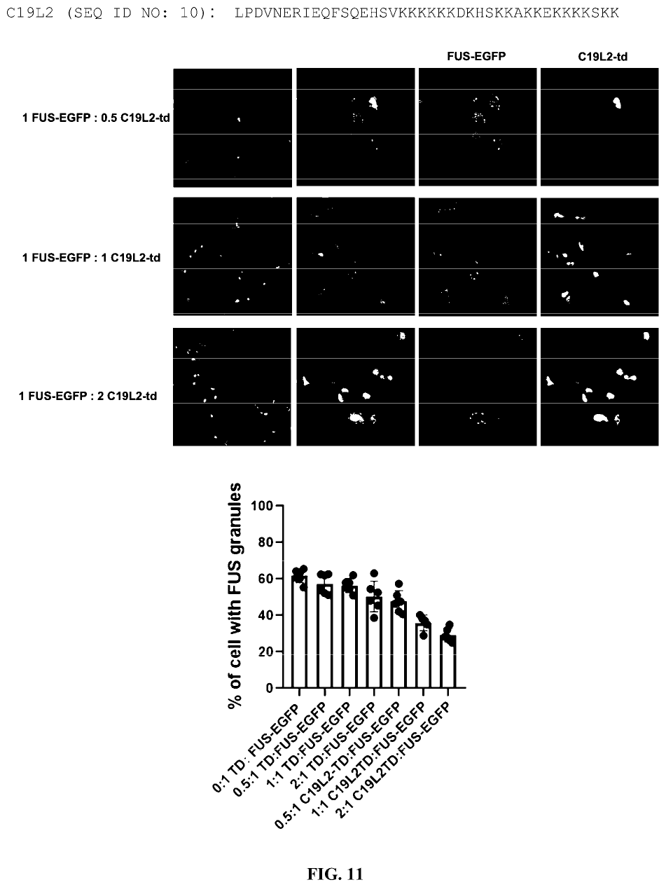

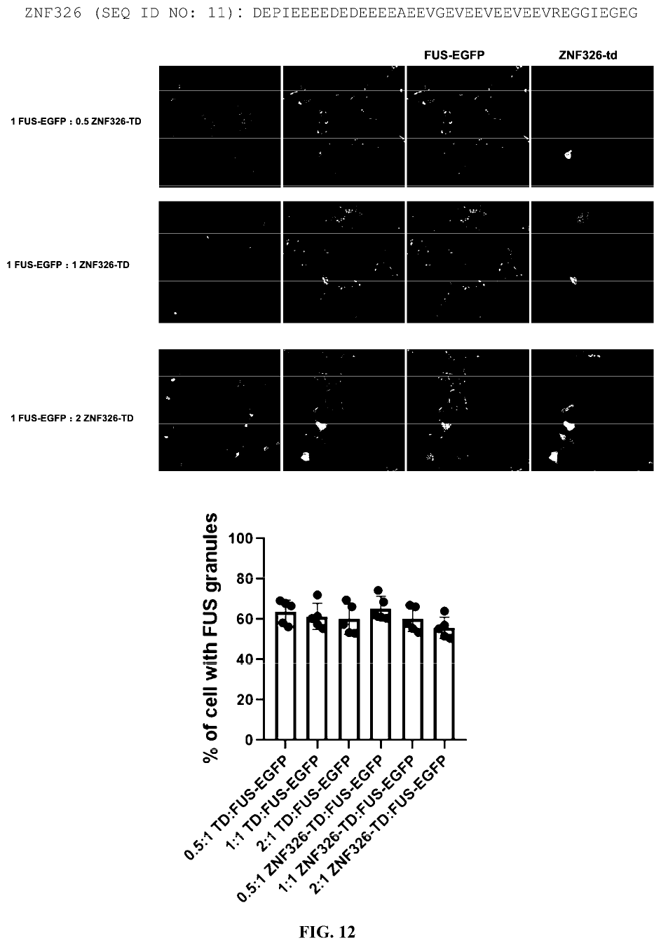

To test the influence of Proline/Glycine residues in depolymerization effect, the inventors selected two identified peptides having few or no P/G: TSYL2 (SEQ ID NO: 9) and C19L2 (SEQ ID NO: 10). As shown in FIGS. 10-11, both selected peptides have depolymerization effect, indicating that P/G residues are not necessary for depolymerization effect.

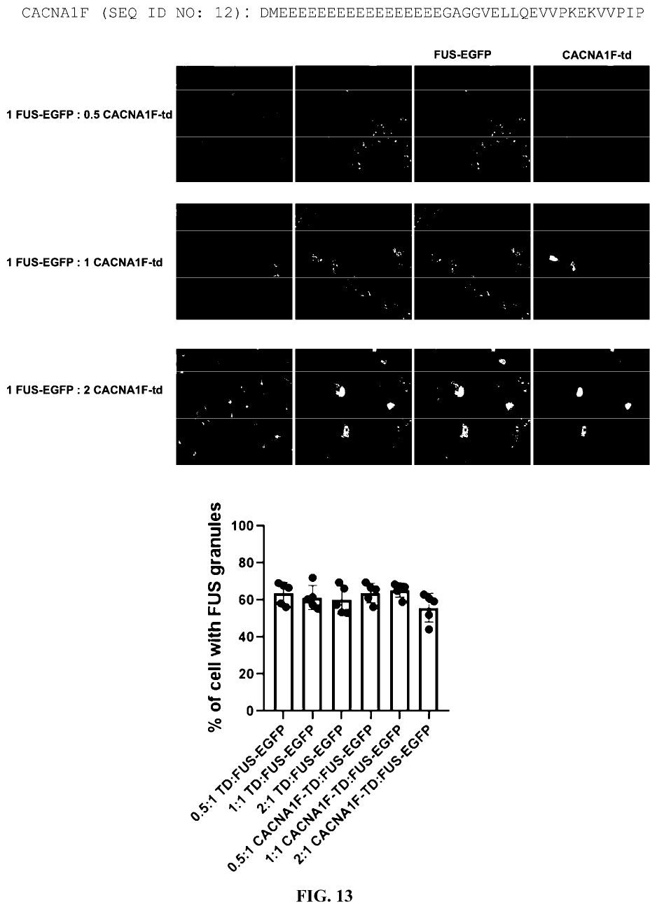

To test the influence of polar residues (S/Q/T/N/Y/C/H) in depolymerization effect, the inventors selected two identified peptides having few or no polar residues: ZNF326 (SEQ ID NO: 11) and CACNA1F (SEQ ID NO: 12). As shown in FIGS. 12-13, both selected peptides have almost no depolymerization effect, indicating that polar residues are necessary for the depolymerization effect.

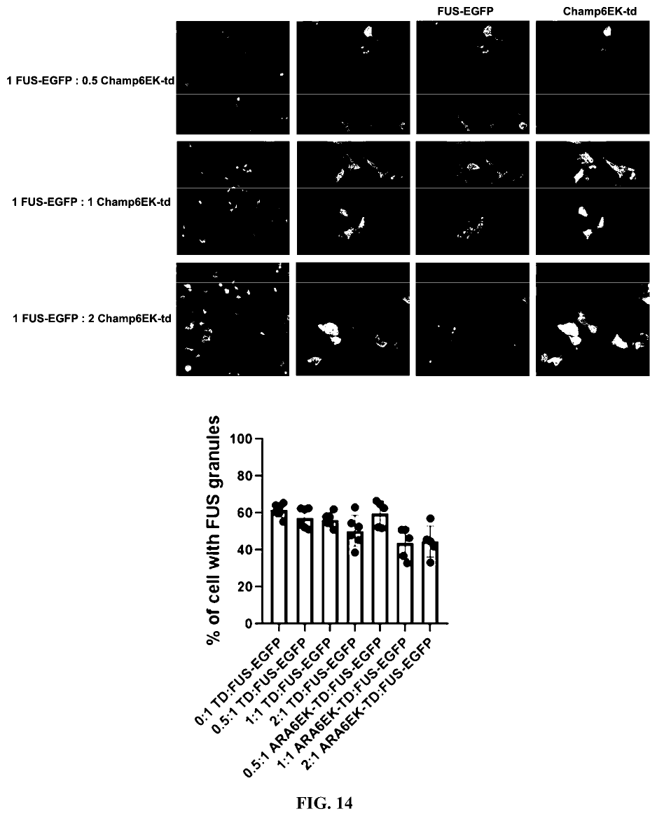

As shown in EXAMPLE 1, when the charged amino acid residues in Champ-E were mutated to a different charged amino acid residue, the variant peptide still has the depolymerization effect. To further test the influence of the charged amino residues, especially when the variant peptide has a mixture of positively and negatively charged amino acid residues, the inventors mutated half of the 12 E residues in Champ-E to positively charged residue K to generate Champ6EK. As shown in FIG. 14, Champ6EK still has certain depolymerization effect.

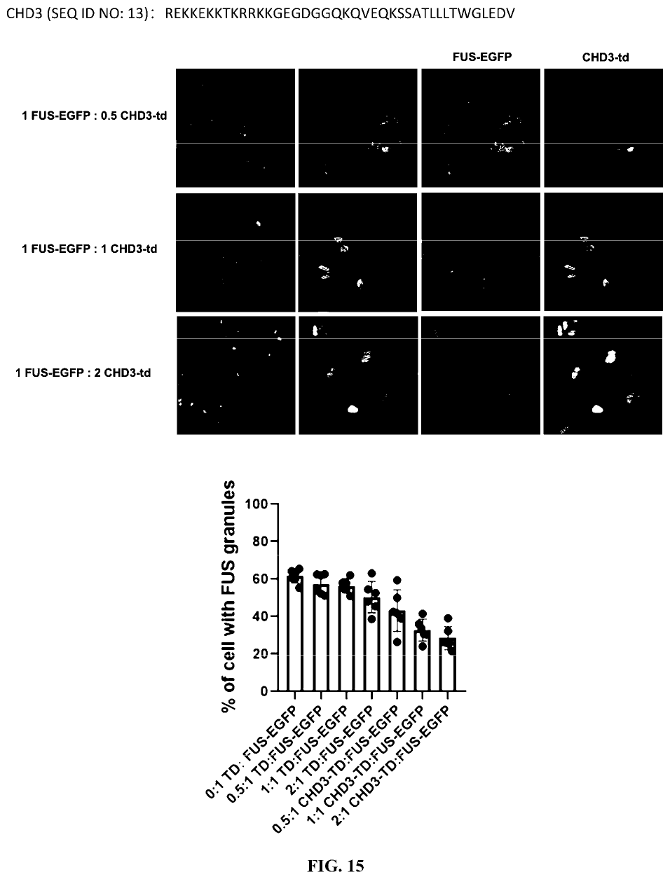

The inventors also selected an identified peptide CHD3 (SEQ ID NO: 13) that has a mixture of positively and negatively charged amino acid residues. CHD3 has charged residues enriched in the N-terminus, with 30% positively charged residues and 27.5% negatively charged residues. As shown in FIG. 15, CHD3 has some depolymerization effect.

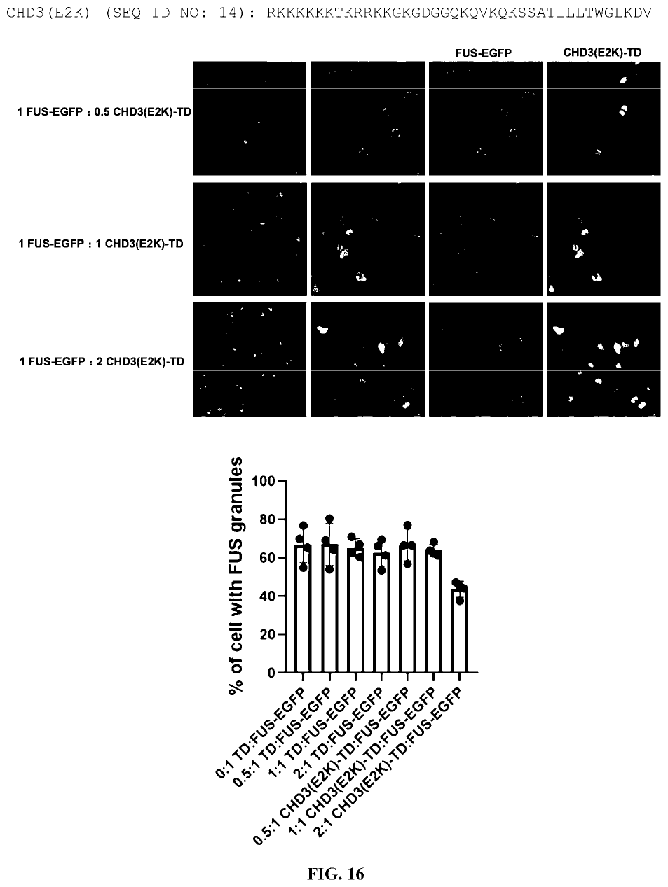

The inventors then mutated the negatively charged residues E in CHD3 to positively charged residues K to generate a variant CHD3(E2K) (SEQ ID NO: 14). As shown in FIG. 16, CHD3(E2K) still has certain depolymerization effect.

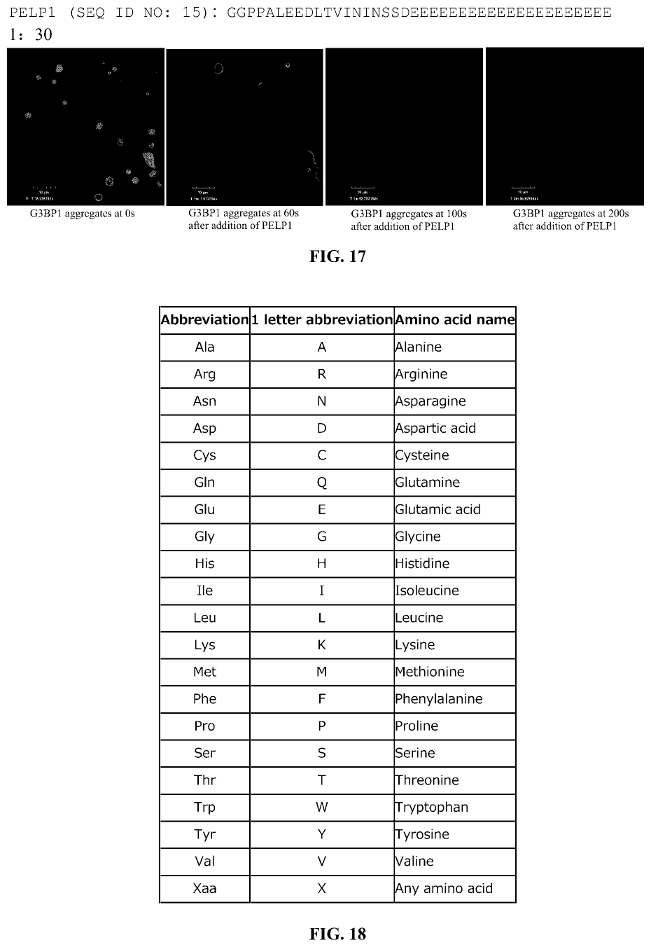

The inventors further tested peptide PELP1 (SEQ ID NO: 15) and found that peptide PELP1 (SEQ ID NO: 15) were capable of dissolving G3BP1 protein aggregates in vitro (FIG. 17).

While the invention has been particularly shown and described with reference to specific embodiments (some of which are preferred embodiments), it should be understood by those having skill in the art that various changes in form and detail may be made therein without departing from the spirit and scope of the present invention as disclosed herein.

What is claimed is:

1. A method of generating a peptide capable of dissolving a protein aggregate, the method comprising:
identifying a naturally occurred protein having a charged amphipathic (Champ) region of 40 to 100 amino acid residues, wherein
(a) at least 30 percent of the amino acid residues in the Champ region are charged amino acid residues selected from Asp, Glu, Arg and Lys, wherein at least 80 percent of the charged amino acid residues are in the N-terminus or C-terminus of the Champ region,
(b) 14 percent to 26 percent of the amino acid residues in the Champ region are hydrophobic amino acid residues selected from Phe, Cys, Leu, Val and Ile, and
(c) at least 10 percent of the amino acid residues in the Champ region are polar amino acid residues selected from Ser, Asn, Thr, Gln, Tyr, Cys and His, and
generating a peptide consisting of the Champ region.

2. The method of claim 1, further comprising:
contacting the peptide with a protein aggregate; and
determining that the peptide dissolves the protein aggregate.

3. The method of claim 1, further comprising mutating any one of the charged amino acid residues in the peptide to a different a charged amino acid residue.

4. The method of claim 1, wherein the peptide is selected from SEQ ID NO: 1-15.

5. A peptide generated according to the method of any one of claims 1-4.

6. A polynucleotide encoding the peptide of claim 5.

7. The polynucleotide of claim 6, which is a DNA or an RNA.

8. A vector comprising the polynucleotide of claim 6 or 7.

9. The vector of claim 8, which is a virus vector.

10. The vector of claim 9, which is an AAV vector.

11. A recombinant virus comprising the polynucleotide of claim 6 or 7.

12. The recombinant virus of claim 11, which is an AAV.

13. A host cell comprising the polynucleotide of claim 6 or 7.

14. A pharmaceutical composition comprising (1) the peptide of claim 5, the polynucleotide of claim 6 or 7, the vector of any one of claims 8-10, the recombinant virus of claim 11 or 12, or the host cell of claim 13, and (2) a pharmaceutically acceptable carrier.

15. A method for dissolving a protein aggregate in a cell, the method comprising introducing into the cell the peptide of claim 5.

16. The method of claim 15, wherein the cell is a neuronal cell.

17. The method of claim 15, wherein the cell is in vitro or in vivo.

18. A method for treating a neurodegeneration disease in a subject in need thereof, the method comprising administering to the subject a therapeutic effective amount of the pharmaceutical composition of claim 14.

19. The method of claim 18, wherein the neurodegeneration disease is selected from frontotemporal dementia, amyotrophic lateral sclerosis, cortical basal ganglia degeneration, Lewy body dementia, Huntington's disease, Lewy body disease, motor neuron disease, frontotemporal degeneration, hippocampal sclerosis, inclusion body myopathy, inclusion body Myositis, Parkinson's disease, argyrophilic granular disease, Alzheimer's disease, Parsons/dementia complex in the Kii Peninsula, progressive supranuclear palsy, and Pick's disease.

20. The method claim 19, wherein the pharmaceutical composition is administered to the central nervous system.

21. The method claim 20, wherein the pharmaceutical composition is administered via spinal cord injection, intrathecal injection, intracerebroventricular injection, intracerebral injection, or intra-hippocampal injection.


SEQUENCE LISTING (15 sequences)

SEQ ID NO: 1 - protein, 42 aa, synthetic construct
MTEPQEESEEEVEEPEERQQTPEVVPDDSGTFYDQTVSNDLE

SEQ ID NO: 2 - protein, 41 aa, synthetic construct
TEPQEESEEEVEEPEERQQTPEVVPDDSGTFYDQTVSNDLE

SEQ ID NO: 3 - protein, 41 aa, synthetic construct
TDPQDDSDDDVDDPDDRQQTPDVVPDDSGTFYDQTVSNDLD

SEQ ID NO: 4 - protein, 41 aa, synthetic construct
TKPQKKSKKKVKKPKKRQQTPKVVPDDSGTFYDQTVSNDLK

SEQ ID NO: 5 - protein, 41 aa, synthetic construct
TQPQQQSQQQVQQPQQRQQTPQVVPDDSGTFYDQTVSNDLQ

SEQ ID NO: 6 - protein, 40 aa, synthetic construct
PMAEEEEEEEDEEEEEEEEEEEDEGPAPPSLYPTVQARPG

SEQ ID NO: 7 - protein, 40 aa, synthetic construct
PCPGAPCCSLVATGSRVPFSGLKEEEEEDGEDDEEEEEEG

SEQ ID NO: 8 - protein, 40 aa, synthetic construct
SNRLMMLRMHNLHGTKPPPSEGSDEEEEEEDEEDEEERKP

SEQ ID NO: 9 - protein, 40 aa, synthetic construct
RSSRRRRRRRRRKQRKVKRESRERNAERMESILQALEDIQ

SEQ ID NO: 10 - protein, 40 aa, synthetic construct
LPDVNERIEQFSQEHSVKKKKKKDKHSKKAKKEKKKKSKK

SEQ ID NO: 11 - protein, 40 aa, synthetic construct
DEPIEEEEDEDEEEEAEEVGEVEEVEEVEEVREGGIEGEG

SEQ ID NO: 12 - protein, 40 aa, synthetic construct
DMEEEEEEEEEEEEEEEEEGAGGVELLQEVVPKEKVVPIP

SEQ ID NO: 13 - protein, 40 aa, synthetic construct
REKKEKKTKRRKKGEGDGGQKQVEQKSSATLLLTWGLEDV

SEQ ID NO: 14 - protein, 40 aa, synthetic construct
RKKKKKKTKRRKKGKGDGGQKQVKQKSSATLLLTWGLKDV

SEQ ID NO: 15 - protein, 40 aa, synthetic construct
GGPPALEEDLTVININSSDEEEEEEEEEEEEEEEEEEEEE